(19) United States
(12) Patent Application Publication
(10) Pub. No.: US 2004/0242612 A1
(43) Pub. Date: Dec. 2, 2004

(54) Use of tyrosine kinase inhibitors for promoting hair growth

(76) Inventors: Alain Moussy, Paris (FR); Jean-Pierre Kinet, Lexington, MA (US)

Correspondence Address: SUGHRUE MION, PLLC, 2100 PENNSYLVANIA AVENUE, N.W., SUITE 800, WASHINGTON, DC, 20037, US

(21) Appl. No.: 10/490,334

(22) PCT Filed: Sep. 20, 2002
(86) PCT No.: PCT/IB02/04290

Related U.S. Application Data
(60) Provisional application No. 60/323,314, filed on Sep. 20, 2001.

(51) Int. Cl. A61K 31/505

(52) U.S. Cl. 514/275

(57) ABSTRACT

The present invention relates to a method for promoting hair growth, preventing or minimizing hair loss comprising administering a tyrosine kinase inhibitor to a human in a need of such treatment, more particularly a non-toxic, selective and potent c-kit inhibitor. Preferably, said inhibitor is unable to promote death of IL-3 dependent cells cultured in presence of IL-3.

The present invention relates to a method for promoting hair growth, preventing or minimizing hair loss comprising administering a tyrosine kinase inhibitor to a human in need of such treatment, more particularly a non-toxic, selective and potent c-kit inhibitor. Preferably, said inhibitor is unable to promote death of IL-3 dependent cells cultured in presence of IL-3.

Hairs are filamentous, keratinized structures consisting of a shaft and a root. The shaft is composed of specialized keratinocytes. The root lies within the hair follicle and comprises the germinative matrix and the keratogenous zone. The germinative matrix consists of pluripotent cells showing mitotic activity giving rise to the hair and its surrounding inner root sheath. Cells arising mitotically from this group move apically and differentiate along several different routes.

Hair growth depends on proliferation of hair follicle matrix cells. It alternates between phases of activity and rest. The anagen phase is a period of growth lasting for two to six years. During this time, the follicle is long and deep, and produces thick, well-pigmented hair. Usually, about 90% of all scalp hairs are in the anagen phase at a given time. This growth phase is followed by the catagen phase for few weeks, which corresponds to the follicle base shrinking. The resting period, or telogen phase, lasts for two to four months. In this phase, the follicle withers even further. Following the telogen phase, the next anagen phase begins, and the old hair is dislodged and falls out to make room for a new hair.

Hair loss (alopecia areata and androgenetic alopecia) is extremely common among healthy adult humans, especially men. Indeed, hair loss affects approximately 50% of men at some point in their lives.

Alopecia androgenetic is an inherited condition, caused by a genetically determined sensitivity to the effects of dihydrotestosterone, which is believed to shorten the anagen phase of the hair cycle, causing miniaturisation of the follicles, and producing progressively finer hairs. Alopecia can also be induced by chemical agents or physical agents (e.g., during anti-cancer chemotherapy), and the condition also results from specific disease states and factors (emotional distress) and with increasing age. Alopecia typically is attributable to a disturbance in the hair renewal cycle leading to acceleration of the frequency of the cycles, which results in a shift in the population of follicles from the anagen phase to telogen. Ultimately, the hair follicles degenerate and a decrease in the number hairs in the affected area of the scalp or skin is observed.

In addition, hair loss can have a serious psychological impact. Studies have shown that hair loss can be associated with low self esteem, depression, introversion, and feelings of unattractiveness. This is reinforced by attitudes in Western society, which place great value on youthful appearance and attractiveness. Some studies have shown that based on appearance alone, men with hair loss are seen as less attractive, less assertive, less likeable, and less successful than men without hair loss.

As a result, there is real need in the development of cosmetic and clinical treatments for promoting hair growth, preventing or minimizing hair loss.

As of today no treatment has been shown to be effective. Mud preparations and plant extracts have been proposed for enhancing hair growth in U.S. Pat. No. 5,798,341, U.S. Pat. No. 5,767,152, U.S. Pat. No. 5,753,713, U.S. Pat. No. 5,750,107, U.S. Pat. No. 5,741,816, U.S. Pat. No. 5,739,111, U.S. Pat. No. 5,723,149, U.S. Pat. No. 5,679,378, U.S. Pat. No. 5,674,497, U.S. Pat. No. 5,663,160, U.S. Pat. No. 5,656,300, U.S. Pat. No. 5,643,898, and U.S. Pat. No. 4,139,619.

2,4-diamino-6-piperidinopyrimidine 3-oxide (minoxidil) and its derivatives have been described in U.S. Pat. No. 4,596,812, EP 522,964, EP 420,707, EP 459,890 and EP 519,819 for slowing hair loss. However, topical application of minoxidil and other agents is only partially effective and suffers from a number of serious side effects in many patients.

Other methods for treating alopecia include:

the administration of anol, anethole and analogs with various mixture of herbs such as umbelliferae, magnoliaceae, labiatae and rutaceae (U.S. Pat. No. 5,422,100),

topical application of retinoic acid to the skin and hair (U.S. Pat. No. 5,514,672),

treatment of hair and scalp which chelating agents, gellan gum, vitamin precursors and derivatives, biotin, γ-linolenic acid, menthol, liposomes, various conditioners, humectants, and folic acid (U.S. Pat. No. 5,523,078),

cosmetic preparations containing solid particles of gold, silver or platinum (U.S. Pat. No. 5,587,168).

and compositions containing others compounds such as estrogens, sulfur, sulfide ions, vasodilators, inorganic selenium compounds, amino acids and protein extracts, garlic powder, brewer's yeast, grapefruit juice, acetic acid and kelp (U.S. Pat. No. 5,629,002 and U.S. Pat. No. 5,674,510).

Despite the above mentioned numerous approaches, effective solutions for stimulating hair growth and for preventing or minimizing hair loss continue to remain elusive.

Numerous factors affect the hair growth cycle including heredity, hormonal deficiencies or imbalances, diet, stress, chemotherapy or aging. For example, it has been shown that various growth factors, steroid hormones, dermo-epithelial interaction, dermal vascularity, neuroectodermal factors, and the immune system are implicated; Stenn et al., Dermatol. Clin., 14, 167-96 (1996) and Lindler et al., Am. J. Pathol., 151, 1601-17 (1997).

More recently, alopecia areata has been considered to be a T-cell mediated autoimmune disease of the hair follicle Freyschmidt-Paul P et al, Curr Pharm Des 2001 February; 7(3):213-30. In accordance with this mechanism, possible future therapeutic approaches include application of immunosuppressive cytokines like TGF-β and IL-10.

Ruckert R. et al, Br J Dermatol 2000 November; 143(5):1036-9 also observed that hair loss following skin inflammation may in part be mediated by keratinocyte (KC) apoptosis. The effects of different cytokines or other apoptosis stimulating agents such as interferon IFN-γ or TNF-α on KC apoptosis have been demonstrated in vitro and in vivo.

Furthermore, histological investigations have shown that in C57BL mice and +/+mice, the number of dermal mast cells in the bald areas was greater than that in controls.

Translation of these data to the human condition is rendered difficult by the complexity of the mechanisms involved in the “classical male pattern alopecia”. Nevertheless, it has been found that the number of inflammatory infiltrates around the follicular infundibula of the alopecic vertices and non-alopecic occiputs of male pattern alopecia patients are significantly greater than the corresponding control value. Of interest, the number of mast cells in the widened fibrous tracts in the vertices of male pattern alopecia patients was found significantly greater than those in the adventitial fibrotic sheaths of control subjects and the non-alopecic occiputs of male pattern alopecia patients.

Mast cells (MC) are tissue elements derived from a particular subset of hematopoietic stem cells that express CD34, c-kit and CD13 antigens (Kirshenbaum et al, Blood. 94: 2333-2342, 1999 and Ishizaka et al, Curr Opin Immunol. 5: 937-43, 1993). Immature MC progenitors circulate in the bloodstream and differentiate in tissues. These differentiation and proliferation processes are under the influence of cytokines, one of utmost importance being Stem Cell Factor (SCF), also termed Kit ligand (KL), Steel factor (SL) or Mast Cell Growth Factor (MCGF). SCF receptor is encoded by the protooncogene c-kit, that belongs to type III receptor tyrosine kinase subfamily (Boissan and Arock, J Leukoc Biol. 67: 135-48, 2000). This receptor is also expressed on others hematopoietic or non hematopoietic cells. Ligation of c-kit receptor by SCF induces its dimerization followed by its transphosphorylation, leading to the recruitement and activation of various intracytoplasmic substrates. These activated substrates induce multiple intracellular signaling pathways responsible for cell proliferation and activation (Boissan and Arock, 2000). Mast cells are characterized by their heterogeneity, not only regarding tissue location and structure but also at the functional and histochemical levels (Aldenborg and Enerback., Histochem. J. 26: 587-96, 1994; Bradding et al. J. Immunol. 155: 297-307, 1995; Irani et al, J. Immunol. 147: 247-53, 1991; Miller et al, Curr Opin Immunol. 1: 637-42, 1989 and Welle et al, J Leukoc Biol. 61: 233-45, 1997).

In connection with the invention, it is proposed that mast cells play a crucial role in alopecia, in that they produce a large variety of mediators categorized here into three groups:

preformed granule-associated mediators (histamine, proteoglycans, and neutral proteases),

lipid-derived mediators (prostaglandins, thromboxanes and leucotrienes),

and various cytokines (IL-1, IL-2, IL-3, IL-4, IL-5, IL-6, IL-8, TNF-a, GM-CSF, MIP-1a, MIP-1b and IFN-γ).

Then, liberation by activated mast cells of mediators (TNF-a, leucotrienes, prostaglandines etc. . . . ) can induce inflammation around follicles leading to apoptosis of cells in the germinative matrix. This support that the inflammatory process mediated by mast cells is, at least in part, responsible for the development of alopecia. Local therapeutic strategies aiming at blocking the activation and the survival of mast cells, for instance through inhibition of c-kit or c-kit signaling can thus be beneficial and could help to decrease hair-loss in such condition.

More specifically, the present invention proposes to use c-kit specific kinase inhibitors to inhibit mast cell proliferation, survival and activation. A new route for promoting hair growth, preventing or minimizing hair loss is provided, which consists of destroying mast cells playing a role in the apoptosis of cells in the hair follicles. It has been found that tyrosine kinase inhibitors and more particularly c-kit inhibitors are especially suited to reach this goal.

### DESCRIPTION

The present invention relates to a method for promoting hair growth, preventing or minimizing hair loss comprising administering a tyrosine kinase inhibitor to a human in need of such treatment.

Tyrosine kinase inhibitors are selected for example from bis monocyclic, bicyclic or heterocyclic aryl compounds (WO 92/20642), vinylene-azaindole derivatives (WO 94/14808) and 1-cycloproppyl-4-pyridyl-quinolones (U.S. Pat. No. 5,330,992), Styryl compounds (U.S. Pat. No. 5,217,999), styryl-substituted pyridyl compounds (U.S. Pat. No. 5,302,606), seleoindoles and selenides (WO 94/03427), tricyclic polyhydroxylic compounds (WO 92/21660) and benzylphosphonic acid compounds (WO 91/15495), pyrimidine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504, U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714,493, U.S. Pat. No. 5,710,158 and WO 95/15758).

Preferably, said tyrosine kinase inhibitors are unable to promote death of IL-3 dependent cells cultured in presence of IL-3.

In another embodiment, the invention is directed to a method for promoting hair growth, preventing or minimizing hair loss comprising administering a c-kit inhibitor to a human in need of such treatment.

Preferably, said c-kit inhibitor is a non-toxic, selective and potent c-kit inhibitor. Such inhibitors can be selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)quinazolones (U.S. Pat. No. 3,551,427).

So, preferably, the invention relates to a method for promoting hair growth, preventing or minimizing hair loss comprising administering a non toxic, potent and selective c-kit inhibitor which is a pyrimidine derivative, more particularly N-phenyl-2-pyrimidine-amine derivatives of formula I:

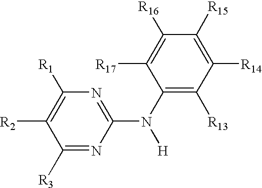

wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II:

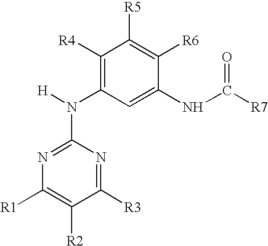

Wherein R1, R2 and R3 are independently chosen from H, F, Cl, Br, I, a C1-C5 alkyl or a cyclic or heterocyclic group, especially a pyridyl group;

R4, R5 and R6 are independently chosen from H, F, Cl, Br, I, a C1-C5 alkyl, especially a methyl group;

and R7 is a phenyl group bearing at least one substituent, which in turn possesses at least one basic site, such as an amino function.

Preferably, R7 is the following group:

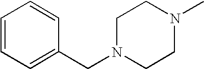

Among these compounds, the preferred are defined as follows:

R1 is a heterocyclic group, especially a pyridyl group,

R2 and R3 are H,

R4 is a C1-C3 alkyl, especially a methyl group,

R5 and R6 are H,

and R7 is a phenyl group bearing at least one substituent, which in turn possesses at least one basic site, such as an amino function, for example the group:

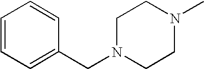

Therefore, in a preferred embodiment, the invention relates to a method for promoting hair growth, preventing or minimizing hair loss comprising the administration of an effective amount of the compound known in the art as CGP57148B:

4-(4-méhylpipérazine-1-ylméthyl)-N-[4-méthyl-3-(4-pyridine-3-yl)pyrimidine-2 ylamino)phényl]-benzamide corresponding to the following formula:

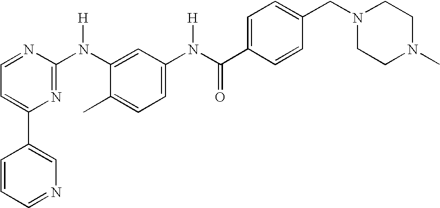

The preparation of this compound is described in example 21 of EP 564 409 and the β-form, which is particularly useful is described in WO 99/03854.

Alternatively, the c-kit inhibitor can be selected from:

indolinone derivatives, more particularly pyrrol-substituted indolinones,

monocyclic, bicyclic aryl and heteroaryl compounds, quinazoline derivatives,

and quinaxolines, such as 2-phényl-quinaxoline derivatives, for example 2-phenyl-6,7-dimethoxy quinaxoline.

In a preferred aspect, the invention contemplated the method mentioned above, wherein said c-kit inhibitor is unable to promote death of IL-3 dependent cells cultured in presence of IL-3.

The expression “hair loss” refers herein to alopecia such as alopecia areata and androgenetic alopecia as well as hair loss caused by other factors. The method depicted above embraces promoting new hair growth, promoting hair growth before, during or after chemotherapy, promoting hair growth in hair transplant patients, preventing, stopping or minimizing hair fall out.

In a further embodiment, c-kit inhibitors as mentioned above are inhibitors of activated c-kit. In frame with the invention, the expression “activated c-kit” means a constitutively activated-mutant c-kit including at least one mutation selected from point mutations, deletions, insertions, but also modifications and alterations of the natural c-kit sequence (SEQ ID N°1). Such mutations, deletions, insertions, modifications and alterations can occur in the transphosphorylase domain, in the juxtamembrane domain as well as in any domain directly or indirectly responsible for c-kit activity. The expression “activated c-kit” also means herein SCF-activated c-kit. Preferred and optimal SCF concentrations for activating c-kit are comprised between 5.10⁻⁷ M and 5.10⁻⁶ M, preferably around 2.10⁻⁶ M. In a preferred embodiment, the activated-mutant c-kit in step a) has at least one mutation proximal to Y823, more particularly between amino acids 800 to 850 of SEQ ID No1 involved in c-kit autophosphorylation, notably the D816V, D816Y, D816F and D820G mutants. In another preferred embodiment, the activated-mutant c-kit in step a) has a deletion in the juxtamembrane domain of c-kit. Such a deletion is for example between codon 573 and 579 called c-kit d(573-579). The point mutation V559G proximal to the juxtamembrane domain c-kit is also of interest.

In this regard, the invention contemplates a method for promoting hair growth, preventing or minimizing hair loss, comprising administering to a mammal in need of such treatment a compound that is a selective, potent and non toxic inhibitor of activated c-kit obtainable by a screening method which comprises:

a) bringing into contact (i) activated c-kit and (ii) at least one compound to be tested; under conditions allowing the components (i) and (ii) to form a complex,

b) selecting compounds that inhibit activated c-kit,

c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild.

Alternatively, in step a) activated c-kit is SCF-activated c-kit wild.

A best mode for practicing this method consists of testing putative inhibitors at a concentration above 10 μM in step a). Relevant concentrations are for example 10, 15, 20, 25, 30, 35 or 40 μM.

In step c), IL-3 is preferably present in the culture media of IL-3 dependent cells at a concentration comprised between 0.5 and 10 ng/ml, preferably between 1 to 5 ng/ml.

Examples of IL-3 dependent cells include but are not limited to:

cell lines naturally expressing and depending on c-kit for growth and survival. Among such cells, human mast cell lines can be established using the following procedures: normal human mast cells can be infected by retroviral vectors containing sequences coding for a mutant c-kit comprising the c-kit signal peptide and a TAG sequence allowing to differentiate mutant c-kits from c-kit wild expressed in hematopoetic cells by means of antibodies.

This technique is advantageous because it does not induce cellular mortality and the genetic transfer is stable and gives satisfactory yields (around 20%). Pure normal human mast cells can be routinely obtained by culturing precursor cells originating from blood obtained from human umbilical vein. In this regard, heparinated blood from umbilical vein is centrifuged on a Ficoll gradient so as to isolate mononucleated cells from other blood components. CD34+ precursor cells are then purified from the isolated cells mentioned above using the immunomagnetic selection system MACS (Miltenyi biotech). CD34+ cells are then cultured at 37° C. in 5% CO₂ atmosphere at a concentration of 10⁵ cells per ml in the medium MCCM (α-MEM supplemented with L-glutamine, penicillin, streptomycin, 5 10⁻⁵ M β-mercaptoethanol, 20% veal foetal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (>98%) is obtained.

It is possible using standard procedures to prepare vectors expressing c-kit for transfecting the cell lines established as mentioned above. The cDNA of human c-kit has been described in Yarden et al., (1987) EMBO J.6 (11), 3341-3351. The coding part of c-kit (3000 bp) can be amplified by PCR and cloned, using the following oligonucleotides:

| | |
|---|---|
| 5′AAGAAGAGATGGTACCTCGAGGGGTGACCC3′ | (SEQ ID No 2) |
| sens | |
| 5′CTGCTTCGCGGCCGCGTTAACTCTTCTCAACCA3′ | (SEQ ID No 3) |
| antisens | |

The PCR products, digested with Not1 and Xho1, has been inserted using T4 ligase in the pFlag-CMV vector (SIGMA), which vector is digested with Not1 and Xho1 and dephosphorylated using CIP (Biolabs). The pFlag-CMV-c-kit is used to transform bacterial clone XL1-blue. The transformation of clones is verified using the following primers:

| | | |
|---|---|---|
| 5′AGCTCGTTTAGTGAACCGTC3′ | (SEQ ID No 4) | sens, |
| 5′GTCAGACAAAATGATGCAAC3′ | (SEQ ID No 5) | antisens. |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit.

Other IL-3 dependent cell lines that can be used include but are not limited to:

BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329.

IC-2 mouse cells expressing either c-kit^{WT} or c-kit^{D814Y} are presented in Piao et al, (1996), Proc. Natl. Acad. Sci. USA 93, 14665-14669.

IL-3 independent cell lines are:

HMC-1, a factor-independent cell line derived from a patient with mast cell leukemia, expresses a juxtamembrane mutant c-kit polypeptide that has constitutive kinase activity (Furitsu T et al, J Clin Invest. 1993;92:1736-1744; Butterfield et al, Establishment of an immature mast cell line from a patient with mast cell leukemia. Leuk Res. 1988;12:345-355 and Nagata et al, Proc Natl Acad Sci USA. 1995;92:10560-10564).

P815 cell line (mastocytoma naturally expressing c-kit mutation at the 814 position) has been described in Tsujimura et al, (1994), Blood 83, 2619-2626.

The extent to which component (ii) inhibits activated c-kit can be measured in vitro or in vivo. In case it is measured in vivo, cell lines expressing an activated-mutant c-kit, which has at least one mutation proximal to Y823, more particularly between amino acids 800 to 850 of SEQ ID No1 involved in c-kit autophosphorylation, notably the D816V, D816Y, D816F and D820G mutants, are preferred.

Example of cell lines expressing an activated-mutant c-kit are as mentioned.

In another preferred embodiment, the method further comprises the step consisting of testing and selecting compounds capable of inhibiting c-kit wild at concentration below 1 μM. This can be measured in vitro or in vivo.

In vivo testing may comprise measuring the ability of the tyrosine kinase inhibitors to alleviate hair loss symptoms in C57BL/Ka mice (cpdm/cpdm) that spontaneously develops alopecia with many of the characteristics of human alopecia (Gijbels M J et al, Am J Pathol 1996 March; 148(3):941-50 and Tanii T et al, Acta Derm Venereol 1985;65(1):64-6).

Therefore, compounds are identified and selected according to the method described above are potent, selective and non-toxic c-kit wild inhibitors.

Alternatively, the screening method as defined above can be practiced in vitro. In this regard, the inhibition of mutant-activated c-kit and/or c-kit wild can be measured using standard biochemical techniques such as immunoprecipitation and western blot. Preferably, the amount of c-kit phosphorylation is measured.

In a still further embodiment, the invention contemplates a method for promoting hair growth, preventing or minimizing hair loss as depicted above wherein the screening comprises:

a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,

b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,

c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

Here, the extent of cell death can be measured by 3H thymidine incorporation, the trypan blue exclusion method or flow cytometry with propidium iodide. These are common techniques routinely practiced in the art.

The method according to the invention includes preventing, delaying the onset of alopecia and/or promoting hair growth in human.

Therefore, the invention embraces the use of the compounds defined above to manufacture a medicament or a cosmetic composition for promoting hair growth, preventing or minimizing hair loss.

The pharmaceutical or cosmetic compositions utilized in this invention may be administered by any number of routes including oral, transdermal, subcutaneous, and topical.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical compositions suitable for use in the invention include compositions wherein c-kit inhibitors are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred. As mentioned above, a tyrosine kinase inhibitor and more particularly a c-kit inhibitor according to the invention is unable to promote death of IL-3 dependent cells cultured in presence of IL-3.

The invention also concerns a pharmaceutical or cosmetic composition for topical administration comprising a tyrosine kinase inhibitor as defined above and optionally at least one compound selected from the group consisting of:

2,4-diamino-6-piperidinopyrimidine 3-oxide (minoxidil) and its derivatives,

anol, anethole and analogs with various mixture of plant extracts such as umbelliferae, magnoliaceae, labiatae and rutaceae,

retinoic acid, chelating agents, gellan gum, vitamin precursors and derivatives, biotin, γ-linolenic acid, menthol, liposomes, various conditioners, humectants, folic acid, particles of gold, silver or platinum (U.S. Pat. No. 5,587,168), estrogens, sulfur, sulfide ions, vasodilators, inorganic selenium compounds, amino acids and protein extracts.

The compositions according to the invention may be presented in all forms normally used for topical application, in particular in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods.

The composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes (paraffin, carnauba, beeswax) may also be used as fatty substances.

As emulsifiers which can be used in the invention, glycerol stearate, polysorbate 60 and the PEG-6/PEG-32/glycol stearate mixture are contemplated.

As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

If desired, a known gelling agent may be added to the composition of the invention. Suitable gelling agents include a synthetic high molecular weight crosslinked polymer of acrylic acid, more specifically an acrylate/C.sub.10-30 alkyl acrylate copolymer available for example under the trade name CARBOMER 1342. Other suitable gelling agents include cellulose and cellulose derivatives such as dihydroxyethyl cellulose (tradename ULTRAGEL).

In addition, a surfactant can be included in the composition so as to provide deeper penetration of the ingredients and of the tyrosine kinase inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art.

For example, compounds with penetration enhancing properties include sodium lauryl sulfate (Dugard, P. H. and Sheuplein, R. J., “Effects of Tonic Surfactants on the Permeability of Human Epidermis: An Electrometric Study,” J. Ivest. Dermatol., V.60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylmethyl sulfoxide (Sekura, D. L. and Scala, J., “The Percutaneous Absorption of Alkylmethyl Sulfides,” Pharmacology of the Skin, Advances In Biolocy of Skin, (Appleton-Century Craft) V. 12, pp. 257-69, 1972). It has been observed that increasing the polarity of the head group in amphoteric molecules increases their penetration-enhancing properties but at the expense of increasing their skin irritating properties (Cooper, E. R. and Berner, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

Suitable solvents include alkyl esters of fatty acids, preferably C.sub.1-12, more preferably C.sub.3-10, alkyl esters of saturated or unsaturated fatty acids containing 8-22 carbon atoms. Particularly preferred solvents include isopropyl myristate, octyl palmitate, WIKENOL 161 (a mixture of esters), etc. Alcohols such as ethanol, propanol, isopropanol, propylene glycol, etc., as well as aqueous mixtures of these alcohols may also be used.

A second class of chemical enhancers are generally referred to as co-solvents. These materials are absorbed topically relatively easily, and, by a variety of mechanisms, achieve permeation enhancement for some drugs. Ethanol (Gale et. al., U.S. Pat. No. 4,615,699 and Campbell et. al., U.S. Pat. Nos. 4,460,372 and 4,379,454), dimethyl sulfoxide (U.S. Pat. Nos. 3,740,420 and 3,743,727, and U.S. Pat. No. 4,575,515), and glycerine derivatives (U.S. Pat. No. 4,322,433) are a few examples of compounds which have shown an ability to enhance the absorption of various compounds.

Any formulation which allows delivery of the active compounds of the present invention to the skin, hair and hair follicles.

Therefore, the invention also contemplates a cosmetic composition comprising a tyrosine kinase inhibitor, more particularly a c-kit inhibitor, and at least one ingredient as depicted above suitable for a topical administration. Preferably, the composition is formulated for the delivery of the tyrosine kinase inhibitor to the skin, hair or hair follicles, such as a hair-conditioning composition.

1. A method for promoting hair growth, preventing or minimizing hair loss, comprising administering a tyrosine kinase inhibitor to a human in need of such treatment.

2. A method according to claim 1, wherein said tyrosine kinase inhibitor is unable to promote death of IL-3 dependent cells cultured in presence of IL-3.

3. A method for promoting hair growth, preventing or minimizing hair loss, comprising administering a c-kit inhibitor to a human in need of such treatment.

4. A method according to claim 3, wherein said c-kit inhibitor is a non-toxic, selective and potent c-kit inhibitor.

5. A method according to claim 4, wherein said inhibitor is selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

6. A method according to claim 4, wherein said inhibitor is selected from the group consisting of:
pyrimidine derivatives, more particularly N-phenyl-2-pyrimidine-amine derivatives.
indolinone derivatives, more particularly pyrrol-substituted indolinones,
monocyclic, bicyclic aryl and heteroaryl compounds,
and quinazoline derivatives.

7. A method according to claim 3, wherein said inhibitor is selected from the group consisting of N-phenyl-2-pyrimidine-amine derivatives having the formula II: 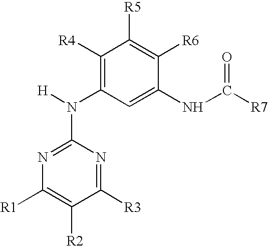
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function, preferably the following group:

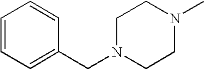

8. A method according to claim 7, wherein said inhibitor is the 4-(4-méhylpipérazine-1-ylméthyl)-N-[4-méthyl-3-(4-pyridine-3-yl)pyrimidine-2 ylamino)phényl]-benzamide.

9. A method according to one of claims 3 to 8, wherein said c-kit inhibitor is unable to promote death of IL-3 dependent cells cultured in presence of IL-3.

10. A method according to one of claims 3 to 9, wherein said c-kit inhibitor is an inhibitor of activated c-kit.

11. A method according to one of claims 3 to 10, wherein said activated c-kit inhibitor is capable of inhibiting SCF-activated c-kit.

12. A method according one of claims 3 to 10, wherein said inhibitor is capable of inhibiting constitutively activated-mutant c-kit.

13. A method for promoting hair growth, preventing or minimizing hair loss, comprising administering to a human in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

14. A method according to claim 13, wherein the screening method further comprises the step consisting of testing and selecting a subset of compounds identified in step b) that are inhibitors of mutant activated c-kit, which are also capable of inhibiting SCF-activated c-kit wild.

15. A method according to claim 13, wherein activated c-kit is SCF-activated c-kit wild in step a).

16. A method according to one of claims 13 to 15, wherein putative inhibitors are tested at a concentration above 10 μM in step a).

17. A method according to one of claims 13 to 16, wherein IL-3 is preferably present in the culture media of IL-3 dependent cells at a concentration comprised between 0.5 and 10 ng/ml, preferably between 1 to 5 ng/ml.

18. A method according to claim 17, wherein IL-3 dependent cells are selected from the group consisting of mast cells, transfected mast cells, BaF3, and IC-2.

19. A method according to one of claims 13 to 18, wherein the extent to which component (ii) inhibits activated c-kit is measured in vitro or in vivo.

20. A method according to one of claims 13 to 18, further comprising the step consisting of testing and selecting compounds capable of inhibiting c-kit wild at concentration below 1 μM.

21. A method according to claim 20, wherein the testing is performed in vitro or in vivo.

22. A method according to one of claims 13 to 21, wherein the inhibition of mutant-activated c-kit and/or c-kit wild is measured using standard biochemical techniques such as immunoprecipitation and western blot.

23. A method according to one of claims 13 to 21, wherein the amount of c-kit phosphorylation is measured.

24. A method according to one of claims 13 to 23, wherein identified and selected compounds are potent, selective and non-toxic c-kit wild inhibitors.

25. A method for promoting hair growth, preventing or minimizing hair loss, comprising administering to a human in need of such treatment a c-kit inhibitor obtainable by a screening method comprising:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being L-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

26. A method according to claim 25, wherein the extent of cell death is measured by 3H thymidine incorporation, the trypan blue exclusion method or flow cytometry with propidium iodide.

27. A method according to one of claims 1 to 26 for treating alopecia such as alopecia areata and androgenetic alopecia as well as hair loss caused by other factors.

28. A method according to one of claims 1 to 26 for promoting new hair growth, promoting hair growth before, during or after chemotherapy, promoting hair growth in hair transplant patients, preventing, stopping or minimizing hair fall out.

29. Use of a c-kit inhibitor to manufacture a medicament or a cosmetic composition for promoting hair growth, preventing or minimizing hair loss.

30. A composition suitable for oral, transdermal, subcutaneous, and topical administration comprising a tyrosine kinase inhibitor, more particularly a c-kit inhibitor for promoting hair growth, preventing or minimizing hair loss.

31. A pharmaceutical or cosmetic composition according to claim 30, which is suitable for topical application.

32. A composition according to claim 31, which is in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type.

33. A composition according to claim 31, which comprises at least one ingredient selected from hydrophilic or lipophilic gelling agents, hydrophilic or lipophilic active agents, emollients, viscosity enhancing polymers, humectants, surfactants, preservatives, antioxidants, solvents, and fillers.

34. A composition according to one of claims 31 to 33, which is formulated for the delivery of the tyrosine kinase inhibitor to the skin, hair or hair follicles.

35. A composition according to claim 34, which is a hair-conditioning composition.


SEQUENCE LISTING (5 sequences)

SEQ ID NO: 1 - protein, 976 aa, Homo sapiens [sequence omitted]

SEQ ID NO: 2 - DNA, 30 bp, Homo sapiens
  Primer
aagaagagatggtacctcgaggggtgaccc

SEQ ID NO: 3 - DNA, 33 bp, Homo sapiens
  Primer
ctgcttcgcggccgcgttaactcttctcaacca

SEQ ID NO: 4 - DNA, 20 bp, Homo sapiens
  Primer
agctcgtttagtgaaccgtc

SEQ ID NO: 5 - DNA, 20 bp, Homo sapiens
  Primer
gtcagacaaaatgatgcaac